(19) United States
(12) Patent Application Publication
(10) Pub. No.: US 2023/0301824 A1
(43) Pub. Date: Sep. 28, 2023

(54) Electric Blanket for Heating Patients

(71) Applicant: Gentherm GmbH, Odelzhausen (DE)

(72) Inventors: Manfred Gies, Odelzhausen (DE); Florian Sandmair, Ried (DE)

(21) Appl. No.: 18/020,660

(22) PCT Filed: Aug. 12, 2021
(86) PCT No.: PCT/DE2021/000133

§ 371 Date: Feb. 10, 2023

(30) Foreign Application Priority Data
Aug. 14, 2020 (DE) 102020004970.2

(51) Int. Cl. A61F 7/00

(52) U.S. Cl. CPC A61F 7/0097; A61F 2007/0071; A61F 2007/0096

(57) ABSTRACT

A medical heating device for heating a person that includes an electronic control unit that is configured as a recyclable module; a flexible heating blanket that is configured to at least partially shield the person from their environment, the flexible heating blanket can be separated from the control unit without destruction, and the flexible heating blanket is configured as a disposable item to permit regular replacement; a heating layer with a carrier layer of a polymeric material and a resistance layer of an electrically conductive material, the heating layer is configured to convert electrical energy into heat, and a plurality of temperature sensors to control the heating layer.

### CROSS REFERENCE TO RELATED APPLICATIONS

This application is a US National Stage of PCT/DE2021/000133 filed on Aug. 12, 2021, which claims priority to DE 10 2020 004 970.2 filed on Aug. 14, 2020, all of which are hereby incorporated by reference herein for all purposes.

### FIELD

The invention relates to a medical heating device for heating a person, with an electronic control unit, which is designed as a recyclable module, and with a flexible heating blanket, which is designed to at least partially shield a person from their environment, wherein the heating blanket can be separated from the control unit without destruction, and wherein the heating blanket is designed as a disposable item in order to permit regular replacement.

The invention further relates to a method of manufacturing a heating device with the steps: Providing an electronic control unit, which is designed as a recyclable module, and providing a flexible heating blanket, which is designed to at least partially shield a person from their environment, wherein the heating blanket can be separated from the control unit without destruction, and wherein the heating blanket is designed as a disposable item in order to permit regular replacement.

Background Generic medical heating devices are used, for example, to heat patients, especially in cases of injury, unconsciousness and surgical operations. The heating blanket of the heating device can be used, for example, to cover a person from above, but also as a base for heating a person from below. This can be advantageous on operating tables. However, the heating blanket of the heating device can also protect unconscious or injured persons from hypothermia during outdoor rescue operations and/or protect unconscious or injured persons from infection with contaminated soil as a sterile separating layer. Additionally or alternatively, the heating blanket of the heating device can be wrapped around the person to be heated.

The electric blanket can cover the person completely or partially.

Heating devices are used particularly in medical care. In this context, increasingly stringent requirements are being placed on the heating devices used in the medical sector. This applies both to the technical properties of the heating device and to its cost-effective manufacturability.

It is desirable to make the heating device inexpensive to manufacture, easy to handle, and available in sterile condition, to the extent possible.

The problem with corresponding heating devices is that there are different requirements for the heating power and its regulation in different areas of application. Up to now, the heating devices do not allow the measurement of the heating power in different zones of the heating blanket and the regulation of the temperature in the respective zone made possible by this measurement.

### SUMMARY

The object of the invention is thus to provide a heating device which ensures that the heating blanket of the heating device safely reaches and does not exceed the desired temperature at any location.

The object is solved by a heating device of the type mentioned above, wherein the heating device comprises a plurality of heating elements, which are arranged to divide the heating blanket into heating zones, and wherein the heating device comprises a plurality of temperature sensors, which are configured to control the heating elements.

The invention makes use of the knowledge that the use of a plurality of heating elements and the division of the heating blanket into heating zones resulting therefrom make it possible to regulate and adjust the temperature of the heating blanket in these zones. It is regularly advantageous to be able to read out the temperature of the heating blanket in different heating zones.

For this, the use of multiple temperature sensors allows the temperature in the individual heating zones to be detected.

The temperature sensors can take the measurement using electrical resistance, emitted thermal radiation, or the change in other physical properties. Examples of suitable temperature sensors are in particular PTC thermistors, pyrometers, bimetal switches or ferromagnetic sensors. The temperature sensors are particularly preferably designed as NTC semiconductor component or as flat measuring resistor made of the same material as the heating conductors of the resistance layer.

Preferably, the control unit is configured to use the read-out temperature data to control the temperature in the heating zones. Particularly preferred is a control unit that allows control of individual heating zones to the extent that the temperature can be regulated independently of the other heating zones. This is regularly advantageous when only certain areas of a person are to be heated.

In a preferred embodiment of the heating device according to the invention, the heating blanket of the heating device comprises the plurality of heating elements and the plurality of temperature sensors. This is particularly preferred because in this case the temperature sensors and heating elements can be effectively protected from external influences, such as heat/cold, humidity or mechanical impact. Accordingly, embodiments in which the heating elements and temperature sensors are part of a heating layer of the heating blanket are particularly preferred. The heating layer is preferably covered by a cover layer to protect the heating layer against moisture, short circuits and mechanical influences.

In another preferred embodiment of the heating device according to the invention, the measurement results of at least one temperature sensor relate to only one part of the heating elements of the heating blanket. The assigned temperature sensors are configured to control the heating power in the respective heating zone.

This enables the detection and control of the temperature in individual areas or heating zones of the heating blanket.

Accordingly, it is particularly preferred that the measurement results of at least one temperature sensor only relate to a specific heating zone.

Preferably, the measurement results of each sensor relate to one heating zone assigned to it in each case. Preferably, the measurement results of each sensor only relate to the heating zone assigned to it in each case.

In another preferred embodiment of the heating device according to the invention, the heating blanket has a heating layer with a carrier layer of a polymeric material and a resistance layer of an electrically conductive material, wherein the heating layer is configured to convert electrical energy into heat.

The polymeric material of the heating layer can be, in particular, polyurethane, polypropylene or polymethylene. The electrically conductive material of the resistive layer can be, in particular, copper, aluminum, nickel, an alloy or a layer composite which includes and/or consists entirely of the aforementioned materials. The resistance layer is preferably structured by selective removal of partial areas in such a way that conductive paths are formed thereon. By their material and/or the width of the paths, these conductive paths are designed to serve as electrical heating resistor or as electrical connection conductor, depending on local needs.

In a particularly preferred embodiment of the heating device according to the invention, the heating layer has at least two heating elements, whose resistance layer is arranged on a common polymeric carrier layer. The heating elements and/or heating resistors can be arranged electrically in series or electrically parallel to each other.

The heating device according to the invention is further advantageously designed in that at least some temperature sensors are assigned to a sensor field, which is designed as a band-like and flexible sheet structure. Preferably, all temperature sensors of the heating blanket are assigned to this sensor field.

In another preferred embodiment of the heating device according to the invention, the sensor field is arranged along the longitudinal axis of the heating blanket and at least partially covers at least part of the heating elements of the heating blanket in order to reliably detect the temperature in the respective heating element.

The sensor field is preferably fixed with an adhesive tape so that the spatial relationship between temperature sensors and the heating element assigned to them is securely maintained even when the heating blanket is folded and crumpled. The sensor field preferably has a sensor carrier made of a polymeric material, preferably the same material as the carrier layer of the heating layer. A conductive layer of an electrically conductive material is preferably applied to the sensor carrier, preferably of the same material as the resistance layer of the heating layer.

The conductive layer of the sensor field is preferably structured by selective removal of partial areas in such a way that sensor connecting conductors are formed thereon.

By their material and/or the width of the paths, these conductive paths are designed to serve as signal conductors. Preferably, at least one temperature sensor has a first sensor connecting conductor for transmitting the measurement results of the temperature sensor to the control unit. Preferably, each temperature sensor has at least one dedicated sensor connecting conductor so that it can distinguish its measurement results from those of other temperature sensors.

It is further preferred to have a heating device according to the invention in which at least one sensor connecting conductor in a sensor field has a deflection in the area of which the distance of the sensor connecting conductor from a longitudinal axis of the heating blanket is different than in another area of its other respective course. In front of and behind the deflection, the sensor connecting conductor runs at a constant distance from the longitudinal axis of the heating blanket. Preferably, the deflection has the shape of a U or a sawtooth.

Particularly preferably, all sensor connecting conductors have at least one deflection at least in the area of the heating element assigned to them.

In another preferred embodiment of the heating device according to the invention, at least one sensor connecting conductor has multiple deflections in its course along the heating blanket. The arrangement of the sensor connecting conductors is preferably selected such that at least one deflection is provided along the heating blanket for each heating element. Preferably, in the area of a deflection, all sensor connecting conductors have coordinated deflections from their remaining course, referred to below as baseline. Preferably, the sensor connecting conductors maintain the same distance between each other in the area of the deflection, which is determined by the distance between their respective baselines. Consequently, the deflection of sensor connecting conductors lying in the direction of the deflection extends over a larger section along the heating blanket than for those sensor connecting conductors lying in the direction away from the deflection. Deflections lying further out are thus more bulbous than those lying further in.

This embodiment is particularly advantageous if the sensor connecting conductors are to be contacted by means of one or more connectors in order to read out the temperature data by means of the control unit. Due to the deflection of the one or more sensor connecting conductors, it is possible to minimize the distance of the sensor connecting conductors in the contacting area of the one or more connectors, so that the dimensions of the one or more connectors can be kept small.

In a particularly preferred embodiment of the heating device according to the invention, the sensor field is an integral part of the resistance layer of the heating layer, wherein the carrier layer of the resistance layer is simultaneously used as a sensor carrier. To ensure that the resulting sensor field is arranged along the longitudinal axis of the heating blanket in this embodiment as well, both the resistance layer and the sensor field can be composed of two elongated submodules.

Furthermore, a heating device according to the invention is preferred, which comprises a plurality of, in particular two, submodules, wherein each submodule has a folding edge and comprises at least one heating element and at least one temperature sensor assigned to the heating element, wherein two submodules with respective folding edges are arranged longitudinally abutting one another. A submodule may be formed by one half of the resistance layer, which comprises the heating elements, and one half of the sensor array, which comprises the temperature sensors. This submodule can be folded along the boundary between the two different functional zones, the longitudinal axis or the folding edge.

In each case, two of the submodules can be arranged with their folding edges longitudinally abutting one another other in such a way that the two halves of the sensor field are arranged towards the same surface of the resulting heating blanket. This is particularly advantageous if the sandwich structure of the heating blanket is not symmetrical to a central position of the heating blanket or if the heating blanket is used under conditions controlled by experts.

In a preferred embodiment of the heating device according to the invention, the sensor fields of the submodules are arranged facing different surfaces of the resulting heating blanket. This is particularly advantageous if the sandwich structure of the heating blanket is symmetrical to a central position of the heating blanket or if the heating blanket is used under conditions that are difficult to supervise. For example, the temperature close to the patient and the temperature in the environment can be matched computationally. Also, it does not matter then which of the heating blanket's two surfaces faces the patient.

The object underlying the invention is further solved by a method of the kind mentioned at the outset, wherein the connecting, in particular the connecting which can be separated without destruction, of the control unit and the heating blanket is effected by means of a connecting cable. The connecting cable allows for arranging the control unit and the heating blanket at a distance from each other, wherein contamination of the heating blanket can be prevented when the control unit is reused. This can be particularly helpful when the control unit is located away from the sickbed or in mobile fashion in a vehicle.

The method according to the invention is advantageously further developed by a reusable adapter provided between the heating blanket and the connecting cable. The reusable adapter is preferably easy to sterilize to prevent contamination of the control unit and/or the connecting cable. The adapter can facilitate the contacting of the heating element. The adapter can contain a circuit board on which electrical functional components are provided for processing signals from the heating blanket to the control unit. The circuit board is preferably designed as a printed circuit board (PCB), preferably on a flexible carrier.

The method according to the invention is preferably further developed by a connector which can be connected to the heating blanket and the adapter and/or the connecting cable and which is configured to allow electrical contacting of sensor connecting conductors of temperature sensors.

### BRIEF DESCRIPTION OF THE DRAWINGS

In the following, preferred embodiments of the invention are explained and described in more detail with reference to the accompanying drawings. The accompanying figures show the following:

FIG. 1 shows an exploded view of a heating device 1 according to the invention with a heating blanket 60;

FIG. 2 shows an exploded view of a heating blanket 60 as shown in FIG. 1;

FIG. 3 shows a top view of a partial section of the resistance layer applied to the heating layer 120 as shown in FIG. 2;

FIG. 4 shows a top and side view of an embodiment of the resistance layer of FIG. 3 with sensor field 110 arranged thereon along the longitudinal axis;

FIG. 5 shows a second embodiment of the sensor field 110;

FIG. 6 shows a third embodiment of the sensor field 110;

FIG. 7 shows connector 70 for the sensor field 110 of FIG. 6;

FIG. 8 shows a top view of a submodule 80a formed from one half of a resistance layer combined with one half of a fourth embodiment of the sensor field 110;

FIG. 9 shows a top view and side view (from the front of the heating blanket) of a plurality of submodules 80a-80d as shown in FIG. 8, each with a folded edge between the sensor field 110 and the resistance layer, and their arrangement along their respective folding edge 81 in alignment with the other submodule 80a-80d; and

FIG. 10. shows a top view and side view (from the front of the heating blanket) of a plurality of submodules 80a-80d as shown in FIG. 8, wherein the two halves of the sensor field 110 face opposite surfaces of the heating blanket 60.

### DETAILED DESCRIPTION

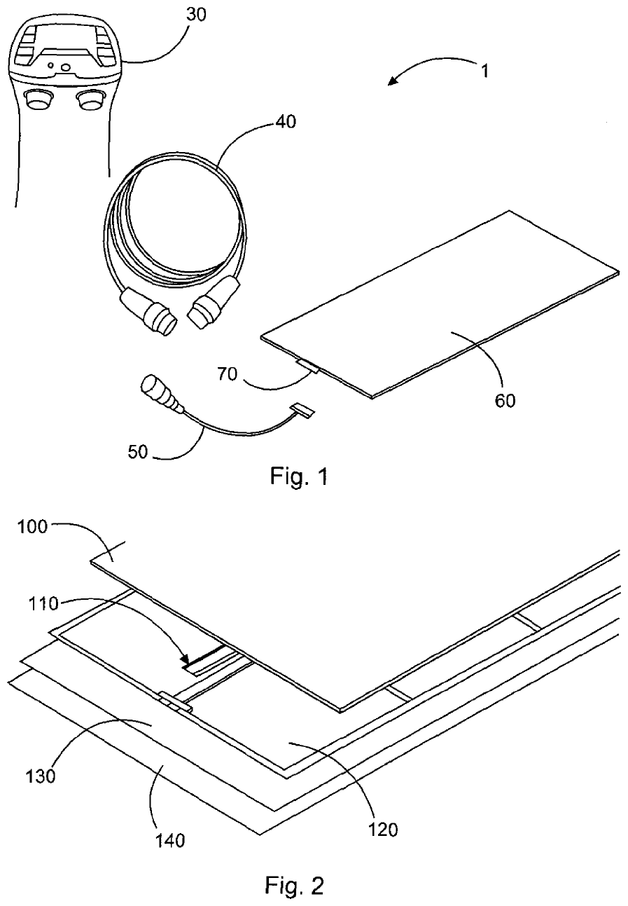

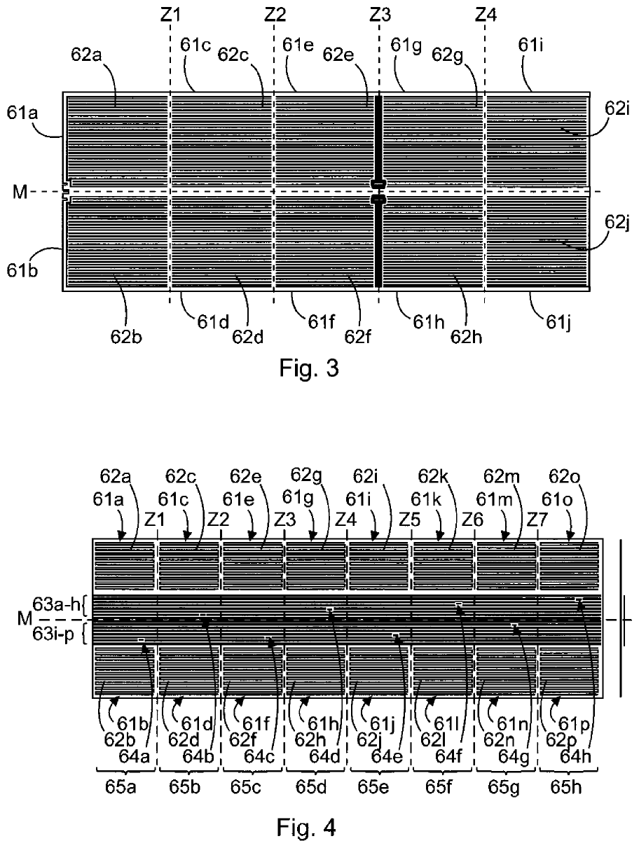

The heating device 1 according to FIG. 1 comprises a flexible heating blanket 60 to at least partially shield a person from their environment. The heating blanket 60 can be used to cover a person from above, but also as a base for heating a person from below. This can be advantageous on operating tables. However, the heating blanket 60 can also protect unconscious or injured persons from hypothermia during outdoor rescue operations and/or protect unconscious or injured persons from infection with contaminated soil as a sterile separating layer. The heating blanket 60 is preferably designed as a low-cost, disposable product to ensure a sterile environment and avoid difficult disinfection after use.

The heating device comprises at least one control unit 30 to monitor the heating power and temperature of the individual heating zones 61a-61p. Since the heating blanket 60 is a disposable product and the control unit 30 is a more complex electronic product, the control unit 30 is expediently not part of the heating blanket 60.

Preferably, the control unit 30 is arranged at a distance from the heating blanket 60 to prevent contamination of the heating blanket 60 when the control unit 30 is reused.

In this embodiment, the control unit 30 and the respective heating blanket 60 are connected to each other via a connecting cable 40. This can be particularly helpful when the control unit is located 30 away from the sickbed or in mobile fashion in a vehicle.

A reusable and easily sterilizable adapter 50 may be provided between the heating blanket 60 and the connecting cable 40 to facilitate contacting of the heating element 62a-62p. Preferably, the adapter contains a printed circuit board (not shown) on which electrical functional components are provided for processing signals from the heating blanket 60 to the control unit 30. The printed circuit board is preferably designed as a printed circuit board (PCB), preferably on a flexible carrier.

As shown in FIG. 2, the heating blanket 60 preferably has a heating layer 120 that is adapted to convert electrical energy into heat.

The heating layer 120 preferably has a carrier layer made of a polymeric material, such as polyurethane, polypropylene, or polymethylene.

Preferably, a resistance layer made of an electrically conductive material, in particular copper, aluminum, nickel, alloys or layered composites containing and/or consisting entirely of the aforementioned materials, is applied to this carrier layer.

The resistance layer is preferably structured by selective removal of partial areas in such a way that conductive paths are formed thereon. By their material and/or the width of the paths, these conductive paths are designed to serve as electrical heating resistor or as electrical connection conductor, depending on local needs.

The heating layer 120 or the resistance layer is preferably covered by a cover layer 100 to protect the heating layer 120 against moisture, short circuits and mechanical influences.

Film or textile material, in particular an impregnated or non-impregnated fleece, are suitable for this purpose.

In addition to this structure, it may be advantageous to provide an insulation layer 130 on the side opposite the resistance layer of the heating layer 120. This results in a cushioning effect of the heating blanket 60 and makes it mechanically easier to handle. It also reduces thermal energy losses and the risk of mechanical stress for the electrical components. More expediently, it is at least partially made of polymer foam, in particular polyethylene (PE).

For its part, the insulation layer 130 may have a protective layer 140 to prevent the absorption of liquids (disinfectants, blood, etc.). A film made of polypropylene, low-density polymethylene (LDPE) or a sandwich of such layers is particularly expedient for this. Preferably, the insulation layer 130 is made of polypropylene with an additional coating of LDPE with a thickness of 0.1-1 μm.

Depending on the intended use of the heating blanket 60, the order of the different layers can also be reversed, e.g. to achieve better protection against the cold when outdoors. Alternatively or in addition to this, one or more layers can be omitted, for example to achieve easier handling or X-ray suitability in the operating room.

The heating blanket 60 has a plurality of heating zones 61a-61p. The object of the invention is thus to provide a heating device which ensures that the heating blanket of the heating device safely reaches and does not exceed the desired temperature at any location. Expediently, the heating blanket 60 according to FIG. 4 is subdivided over its entire surface into heating zones 61a-61p, wherein the size of the heating zones 61a-61p in this embodiment is defined by the zone axes 21-27 or the longitudinal axis M of the heating blanket 60.

At least one of these heating zones 61a-61p has at least one heating resistor, preferably designed as a submodular heating element 62a-62p. Preferably, all heating zones 61a-61p have at least or exactly one heating element 62a-62p.

An exemplary heating element 62a-62p has dimensions of approx. 1,300×500 mm. The exemplary heating element 62a-62p operates at an operating voltage of 10-50 V, preferably 12-24 V. The exemplary heating element 62a-62p has a power density of 10-500 W/m², preferably 50-300 W/m², preferably 150-250 W/m². The exemplary heating element 62a-62p preferably has a power of 10-500 W, preferably 50-200 W, preferably 100-130 W. The resistance is, for example, 1-10 ohms, preferably 2-6 ohms.

Preferably, the heating layer 120 has at least two heating resistors and/or heating elements 62 whose resistance layer is arranged on a common polymeric carrier layer. The heating elements 62a-62p and/or heating resistors can be arranged electrically in series or electrically parallel to each other.

Preferably, the resistance layer of the heating blanket 60 is composed of a plurality of partial sheet structures. As a result, larger and wider heating blankets 60 can be produced without having to manufacture the starting material in oversized dimensions. This would entail high costs for manufacturing equipment and higher scrap. The composition of the resistance layer from a plurality of partial sheet structures regularly proves to be particularly helpful if the total heating surface is composed of integer multiples of heating elements 62a-62p produced by means of roller cliches.

Preferably, the resistance layer is composed of two elongated strips abutting one another along the longitudinal axis of the heating blanket 60.

Preferably, each partial surface has several heating zones 61a-61p. Preferably, a heating element 62a-62p is formed in each of these heating zones 61a-61p.

At least one of the heating zones 61a-61p has at least one temperature sensor 64a-64h assigned to it to control the heating power in this heating zone 61a-61p.

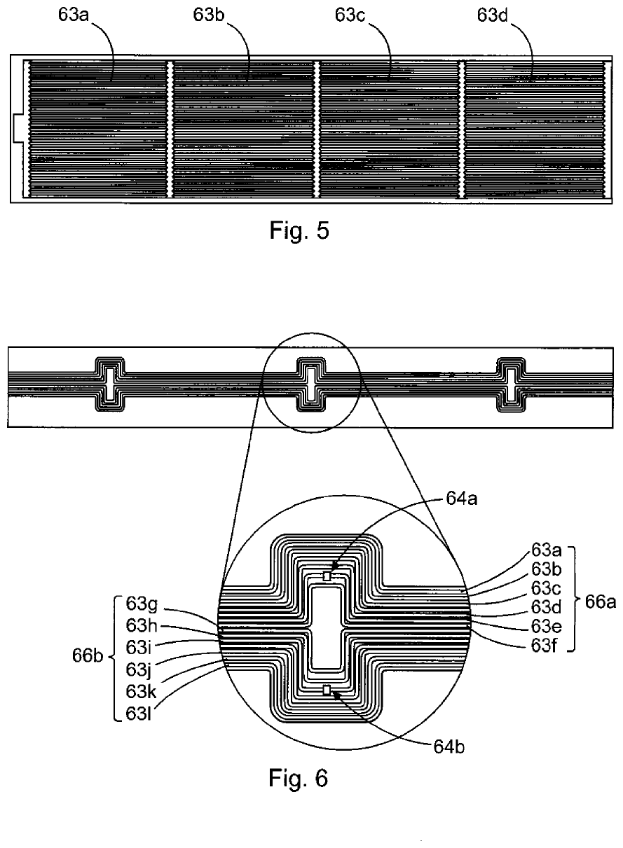

It is preferably designed as an NTC semiconductor component (cf. FIGS. 4, 6, 9 and 10) or as a flat measuring resistor made of the same material as the heating conductors of the resistance layer (cf. FIG. 5).

Preferably, the heating blanket 60 has a plurality of temperature sensors 64a-64h. Preferably, the heating blanket 60 has one temperature sensor 64a-64h per heating zone 61a-61p, per heating element 62a-62p and/or per heating element pair 65a-65h. A heating element pair 65a-65h is formed by two opposing heating elements 62a-62p separated by the longitudinal axis M.

The measurement results of at least one temperature sensor 64a-64h only relate to one part of the heating elements 62a-62p of the heating blanket 60. Preferably, the measurement results of at least one temperature sensor 64a-64h relate only to one specific heating zone 61a-61p. Preferably, the measurement results of each temperature sensor 64a-64h relate to one heating zone 61a-61p assigned to it in each case. Preferably, the measurement results of each temperature sensor 64a-64h relate only to the heating zone 61a-61p assigned to it in each case.

Preferably, at least some temperature sensors 64a-64h are assigned to a band-like and flexible sheet structure, here called sensor field 110. Preferably, all temperature sensors 64a-64h of the heating blanket 60 are assigned to this sensor field 110.

The sensor field 110 is arranged along the longitudinal axis of the heating blanket 60. In this respect, it preferably covers each of the heating elements 62a-62p of the heating blanket 60 at least partially in order to reliably detect the temperature in the respective heating element 62a-62p.

The sensor field 110 is preferably fixed with an adhesive tape so that the spatial relationship between temperature sensors 64a-64h and the heating element 62a-62p assigned to them is securely maintained even when the heating blanket 60 is folded and crumpled.

The sensor field 110 preferably has a sensor carrier made of a polymeric material, preferably the same material as the carrier layer of the heating layer 120.

A conductive layer of an electrically conductive material is preferably applied to the sensor carrier, preferably of the same material as the resistance layer of the heating layer 120.

The conductive layer is preferably structured by selective removal of partial areas in such a way that sensor connecting conductors 63a-63p are formed thereon. By their material and/or the width of the paths, these conductive paths are designed to serve as signal conductors. In an embodiment according to FIG. 5, however, partial sections of this resistance layer can also serve as temperature sensors 64a-64h.

Preferably, at least one temperature sensor 64a-64h has a first sensor connecting conductor 63a-63p for transmitting the measurement results of the temperature sensor 64a-64h to the control unit 30. Preferably, each temperature sensor 64a-64h has at least one dedicated sensor connecting conductor 63a-63p so that it can distinguish its measurement results from those of other temperature sensors 64a-64h.

Preferably, at least one temperature sensor 64a-64h has a second sensor connecting conductor 63a-63p. To output signals, the temperature sensor 64a-64h can generate a potential difference between its two connecting conductors, the magnitude and/or time progression of which represents a coded representation of the sensor measurements.

In this respect, a plurality of temperature sensors 64a-64h could share a common, preferably grounded sensor connecting conductor 63a-63p as a second sensor connecting conductor 63a-63p. This would reduce the number of sensor connecting conductors 63a-63p.

In terms of manufacturing automation, it is preferable that each temperature sensor 64a-64h is assigned two sensor connecting conductors 63a-63p that are uniquely and exclusively allocated to it.

At least one sensor connecting conductor 63a-63p preferably extends over at least 90% of the length of the sensor field 110, preferably over its entire length. Preferably, this applies to all sensor connecting conductors 63a-63p.

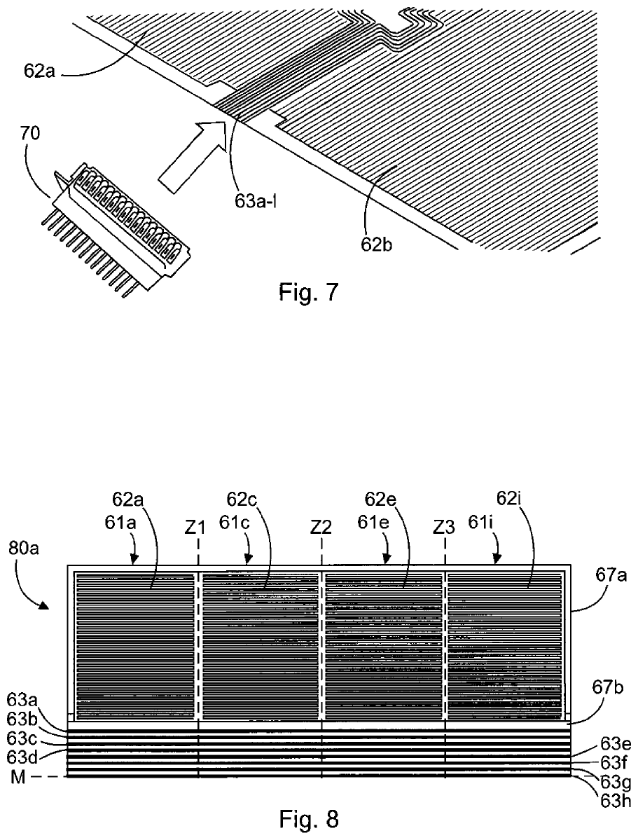

In the embodiments of FIG. 4, and FIGS. 8 to 10, at least one sensor connecting conductor 63a-63p runs parallel to the direction of the course of the sensor field 110, the longitudinal axis of the heating blanket 60 and/or to further sensor connecting conductors 63a-63p over at least 90% of its entire length. Preferably, this proportion is 100%, since such embodiments can be manufactured from continuous roll material, for example.

In the embodiments of FIG. 4, and FIGS. 6 to 10, the placement of at least one temperature sensor 64a-64h is such that it makes electrical contact with two sensor connecting conductors 63a-63p arranged directly adjacent to one another. This can be done in particular by soldering, crimping or using electrically conductive adhesives. Since each temperature sensor 64a-64h in these embodiments has its own sensor connecting conductor 63a-63p, each temperature sensor 64a-64h can have an individual position different from all other temperature sensors 64a-64h with respect to the width of the heating blanket 60 as a coordinate axis.

In the embodiments of FIGS. 6 and 7, the sensor connecting conductors 63a-63p are arranged as close as possible to the longitudinal axis of the heating blanket 60. This allows simple electrical contacting of all sensor connecting conductors 63a-63p by means of a compact connector 70 as shown in FIG. 7. For this purpose, preferably at least one sensor connecting conductor 63a-63p runs over at least 40% of its length parallel to the direction of the course of the sensor field 110, the longitudinal axis of the heating blanket 60 and/or to further sensor connecting conductors 63a-63p. Preferably, this proportion is at least 50%, more preferably 60%. This course is referred to below as the baseline of the respective sensor connecting conductor 63a-63p.

Preferably, in a sensor field 110 according to FIGS. 6 and 7, at least one sensor connecting conductor 63a-63p has a deflection in the area of which the distance of the sensor connecting conductor 63a-63p from the longitudinal axis M of the heating blanket 60 is different than in the area of its respective baseline. In front of and behind the deflection, the sensor connecting conductor 63a-63p runs at a constant distance from the longitudinal axis M of the heating blanket 60. Preferably, the deflection has the shape of a U or a sawtooth. Preferably, all sensor connecting conductors 63a-63p have at least one deflection at least in the area of the heating element 62a-62p assigned to them.

Preferably, at least one sensor connecting conductor 63a-63p has multiple deflections in its course along the heating blanket 60. Their arrangement is preferably selected such that at least one deflection is provided along the heating blanket 60 for each heating element 62a-62p.

If heating elements 62a-62p are arranged in pairs as in FIG. 4, each with a heating element 62a, 62c, 62e, 62g, 62i, 62k, 62m, 62o above and 62b, 62d, 62f, 62h, 62j, 621, 62n, 62p below the longitudinal axis M of the heating blanket 60, the term “heating element” in the preceding paragraph refers to such a pair of heating elements 65a-65h. In this case, sensor connecting conductors 63a-63p are preferably arranged in two sensor connecting conductor groups 66a, 66b, wherein the first sensor connecting conductor group 66a is assigned to the heating elements 62a-62p arranged above, and the second sensor connecting conductor group 66b is assigned to the heating elements 62a-62p arranged below the longitudinal axis M of the heating blanket 60. The deflections of the two sensor connecting conductor groups 66a, 66b are preferably arranged mirror-symmetrically to one another here, with the mirroring taking place along the longitudinal axis M of the heating axis, so that the deflections of the two sensor connecting conductor groups 66a, 66b point in directions opposite to one another.

Preferably, in the range of a deflection, all sensor connecting conductors 63a-63p have deflections coordinated with each other from their respective baselines. Preferably, they maintain the same distance between each other in the area of the deflection, which distance is determined by the distance between their respective baselines.

Consequently, the deflection of sensor connecting conductors 63a-63p lying in the direction of the deflection extends over a larger section along the heating blanket 60 than for those sensor connecting conductors 63a-63p lying in the direction away from the deflection. Deflections lying further out are thus more bulbous than those lying further in.

Temperature sensors 64a-64h can be arranged in the area of the deflections. Preferably, for each heating element 62a-62p, respectively one temperature sensor 64a-64h is arranged in the deflection assigned to the heating element 62a-62p. As described, each sensor is assigned to two sensor connecting conductors 63a-63p that are individually assigned to it.

The embodiments of FIGS. 4 to 7 show that the sensor field 110 can be designed separately from the resistance layer, and thus can be arranged on a plane of the heating blanket 60 that is different therefrom.

The further embodiments of FIGS. 8 to 10 show that the sensor field 110 can also be an integral part of the resistance layer. For this, the carrier layer of a resistance layer is simultaneously used as a sensor carrier.

To ensure that the resulting sensor field 110 is arranged along the longitudinal axis M of the heating blanket 60 in this case as well, both the resistance layer and the sensor field 110 are composed of two elongated submodules 80a, 80b.

In one embodiment, a submodule 80a is first manufactured as shown in FIG. 8. It is formed from one half of the resistance layer and one half of the sensor field 110, respectively. This module can be folded along the boundary between the two different functional zones, the longitudinal axis M. The electrically active surfaces preferably face towards surfaces of the resulting two-layer structure that face away from one another.

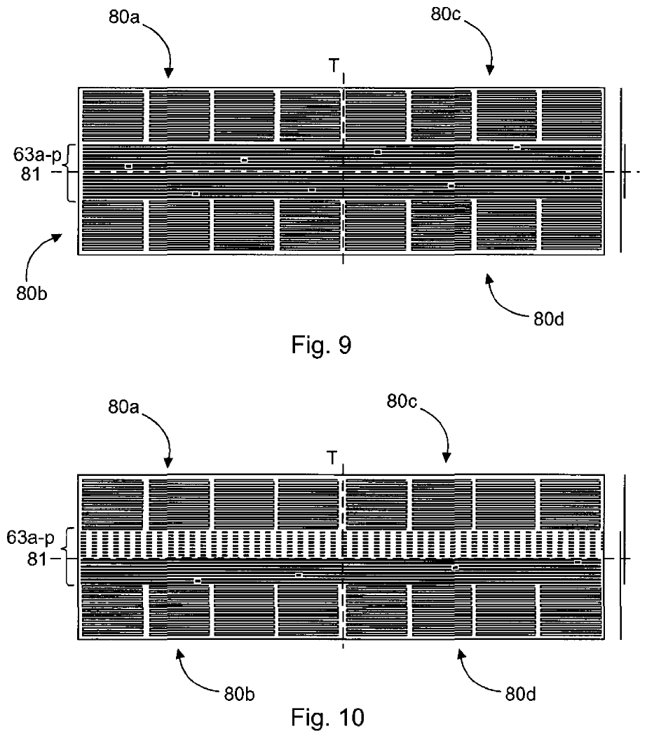

In the embodiment according to FIG. 9, an arrangement with 4 submodules 80a-80d is shown, which are separated by the submodule axis T and the folding edge 81. In each case, two of these submodules 80a and 80b or 80c and 80d are arranged with their folding edges 81 longitudinally abutting one another other in such a way that the two halves of the sensor field 110 are arranged towards the same surface of the resulting heating blanket 60.

This is particularly advantageous if the sandwich structure of the heating blanket 60 is not symmetrical to a central position of the heating blanket or if the heating blanket 60 is used under conditions controlled by experts.

In the embodiment shown in FIG. 10, two of these submodules 80a and 80b or 80c and 80d are arranged with their folding edges longitudinally abutting one another other in such a way that the two halves of the sensor field 110 are arranged facing different surfaces of the resulting heating blanket 60. This is advantageous, particularly if the sandwich structure of the heating blanket 60 is symmetrical to a central position of the heating blanket 60 or if the heating blanket 60 is used under conditions that are difficult to supervise. For example, the temperature close to the patient and the temperature in the environment can be matched computationally. Also, it does not matter then which of the heating blanket's 60 two surfaces faces the patient.

### REFERENCE NUMBERS

1 Heating device
30 Control unit
40 Connecting cable
50 Adapter
60 Heating blanket
61a-61p Heating zone
62a-62p Heating element
63a-63p Sensor connecting conductor
64a-64h Temperature sensor
65a-65h Pair of heating elements
66a, 66b Sensor connecting conductor group
67a, 67b Collecting electrodes
70 Connector
80a-80d Submodule
81 Folding edge
100 Cover layer
110 Sensor field
111a, 111b Half of the sensor field
120 Heating layer
130 Insulation layer
140 Protective layer
M Longitudinal axis
Z1-Z7 Zone axis
T Submodule axis

1.-15. (canceled)

16. A medical heating device for heating a person, comprising:
an electronic control unit that is configured as a recyclable module;
a flexible heating blanket that is configured to at least partially shield the person from an environment surrounding the person, the flexible heating blanket is configured to be separable from the control unit without destruction of the flexible heating blanket, and the flexible heating blanket is configured as a disposable item to permit regular replacement;
a heating layer with a carrier layer of a polymeric material and a resistance layer of an electrically conductive material, the heating layer is configured to convert electrical energy into heat, and
a plurality of temperature sensors to control the heating layer.

17. The heating device according to claim 16, wherein the heating blanket comprises a plurality of heating elements.

18. The heating device according to claim 16, wherein a measurement result from the plurality of temperature sensors only relates to a portion of the heating element.

19. The heating device according to claim 16, wherein the heating layer has at least two heating elements whose resistance layer is arranged on a common polymeric carrier layer.

20. The heating device according to claim 16, wherein at least some of the plurality of temperature sensors are assigned to a sensor field, which is configured as a band-like and flexible sheet structure.

21. The heating device according to claim 20, wherein the sensor field is arranged along a longitudinal axis of the heating blanket and at least partially covers at least part of the heating element to detect a temperature in the heating element.

22. The heating device according to claim 16, wherein at least one sensor connecting conductor in a sensor field has a deflection in an area of which a distance of the sensor connecting conductor from a longitudinal axis of the heating blanket is different than in another area of its other respective course.

23. The heating device according to claim 16, wherein at least one sensor connecting conductor has a plurality of deflections in its course along the heating blanket.

24. The heating device according to claim 20, wherein the sensor field is an integral part of the resistance layer of the heating layer, wherein the carrier layer of the resistance layer is simultaneously used as a sensor carrier.

25. A medical heating device for heating a person comprising
an electronic control unit that is configured as a recyclable module;
a flexible heating blanket that is configured to at least partially shield the person from an environment surrounding the person, the flexible heating blanket is configured to be separable from the control unit without destruction of the flexible heating blanket, and the flexible heating blanket is configured as a disposable item to permit regular replacement;
a plurality of heating elements that are arranged to divide the heating blanket into heating zones, and
a plurality of temperature sensors, which are arranged to control the heating elements.

26. The heating device according to claim 25, wherein the heating blanket has a heating layer with a carrier layer of a polymeric material and a resistance layer of an electrically conductive material, the heating layer is configured to convert electrical energy into heat.

27. the heating device according to claim 26, wherein the heating layer has at least two heating elements whose resistance layer is arranged on a common polymeric carrier layer, wherein at least some of the plurality of temperature sensors are assigned to a sensor field, which is configured as a band-like and flexible sheet structure, wherein the sensor field is arranged along a longitudinal axis of the heating blanket and at least partially covers at least part of the heating element to detect a temperature in the heating element, wherein at least one sensor connecting conductor has a plurality of deflections in its course along the heating blanket, wherein the sensor field is an integral part of the resistance layer of the heating layer, wherein the carrier layer of the resistance layer is simultaneously used as a sensor carrier.
